(19) United States
(12) Patent Application Publication
(10) Pub. No.: US 2011/0112285 A1
(43) Pub. Date: May 12, 2011

(54) NOVEL METHOD FOR PREPARING OLIGONUCLEOTIDES COMPRISING A 5-PHOSPHATE MONOESTER OR 5-THIOPHOSPHATE MONOESTER END

(75) Inventors: Eric Derfrancq, Saint Pierre D'Allevard (FR); Remi Lartia, Voiron (FR)

(73) Assignee: UNIVERSITE JOSEPH FOURIER, Grenoble Cedex 09 (FR)

(21) Appl. No.: 12/988,220

(22) PCT Filed: Apr. 15, 2009
(86) PCT No.: PCT/FR09/50689

§ 371 Date: Jan. 7, 2011

(30) Foreign Application Priority Data
Apr. 16, 2008 (FR) 08/02097

(51) Int. Cl. C07H 1/00

(52) U.S. Cl. 536/25.3

(57) ABSTRACT

The preparation of oligonucleotides having a 5′-phosphate monoester end.

The present invention relates to the preparation of oligonucleotides comprising a 5′-phosphate monoester or 5′-thiophosphate monoester end.

The oligonucleotides comprising a 5′-phosphate end are compounds used, inter alia, in molecular biology in the context of enzymatic ligation or the study of nucleic acid lesion repair mechanisms, and in therapy as adjuvants for vaccine preparations using CpG oligonucleotides or for the inhibition of gene expression by the siRNAs.

The 5′-phosphate oligonucleotides are in general obtained by chemical synthesis by the introduction of a phosphate group in the 5′ position by means of phosphorylation reagents belonging to the family of phosphoramidites described in the references below (Solid phase 5′-phosphorylation of oligonucleotides, Connolly B. A., Tetrahedron Lett., 1987, 28, 463-466; Chemical 5′-phosphorylation of oligonucleotides valuable in automated DNA synthesis, Uhlmann E., Engel J., Tetrahedron Lett., 1986, 27, 1023-1026; A chemical 5′-phosphorylation of oligonucleotides that can be monitored by trityl cation release, Horn T., Urdea M. S., Tetrahedron Lett., 1986, 27, 4705-4708; Synthesis and utility of a DNA phosphorylating agent based on 2-(triphenylsilyl)ethanol, Celebuski J. E., Chan C., J. Org. Chem., 1992, 57, 5535-5538; A new approach for chemical phosphorylation of oligonucleotides at the 5′-terminus, Guzaev A., Salo H., Azhayev A, Lönnberg H., Tetrahedron, 1995, 51, 9375-9384; A novel reagent for the chemical phosphorylation of oligonucleotides, Leuck M., Vagle K. E., Roach J. S., Wolter A., Tetrahedron Lett., 2004, 45, 321-324; New reagent for the preparation of oligonucleotides involving a 5′-thiophosphate or a 5′ phosphate group, Lartia R., Asseline U., Tetrahedron Lett., 2004, 45, 5949-5952; An efficient reagent for the phosphorylation of deoxyribonucleotides, DNA oligonucleotides, and their thermolytic analogues, Ausin C., Grajkowski A., Cieslak J., Beaucage S. L., Org. Lett., 2005, 7, 4201-4204; A versatile reagent for the synthesis of 5′-phosphorylated, 5′-thiophosphorylated or 5′-phosphoramidate conjugated oligonucleotides, Meyer A., Bouillon C., Vidal S., Vasseur J.-J., Morvan F., Tetrahedron Lett., 2006, 47, 8867-8871; Solid-phase synthesis of terminal oligonucleotide-phosphoramidate conjugates; Cooke L. A., Frauendorf C., Gilea M. A., Holmes S. C., Vyl J. S., Tetrahedron Lett., 2006, 47, 719-722.

However, all these different phosphoramidite-type reagents have the drawbacks inherent to this chemical family, namely:
a time-consuming and tedious manufacturing process,
a high price when the reagent is available commercially,
a high sensitivity to water and oxidants such as atmospheric oxygen, making it necessary to handle the reagent under an inert atmosphere,
a short shelf life of the reagent, quite particularly when the packaging has been opened,
presentation in the form of oil, which makes handling difficult in particular when weighing small quantities.

One of the aspects of the invention relates to a method which makes it possible to obtain oligonucleotides comprising a 5′-phosphate monoester or 5′-thiophosphate monoester end, without having recourse to phosphoramidite-type reagents with their associated drawbacks.

Another aspect of the invention is providing a simple method for the synthesis of oligonucleotides comprising a 5′-phosphate monoester or 5′-thiophosphate monoester end.

Yet another aspect of the invention allows the provision of a method for the synthesis of oligonucleotides comprising a molecule of interest such as fluorescent probes, peptides, sugars, intercalating agents etc. in the 5′ position.

The present invention relates to the use of at least one oxidant in particular chosen from:
the hypervalent iodine derivatives, in particular 2-iodoxybenzoic acid (IBX) and Dess-Martin periodinane (DMP), and
Swern's reagent,
   for the synthesis of oligonucleotides, modified or not, in particular of oligoribonucleotides, oligodeoxyribonucleotides, said oligonucleotides comprising a 5′-phosphate monoester or 5′-thiophosphate monoester end.

By the expression “hypervalent iodine derivatives”, is meant iodine compounds in which the iodine atom contains more than eight electrons in the valence shell required by the octet rule. The hypervalent iodine derivatives can be hypervalent iodine (III) derivatives referred to as iodanes, or hypervalent iodine (V) derivatives referred to as periodinanes, and allow the oxidation of primary alcohols to aldehydes and of secondary alcohols to ketones.

Numerous hypervalent iodine (III) derivatives exist such as, but without being limited to these: PIFA (Bis(trifluoroacetoxy)iodobenzene, PIDA (diacetoxy)iodobenzene,

Koser's reagent:

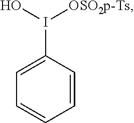

IBA (iodosyl benzoic acid) derivatives:

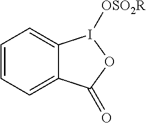

with R=p-Ts or Ms. Preferred compounds of hypervalent iodine (V) derivatives, without being limited to these,

### are IBX:

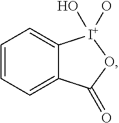

and Dess Martin (DMP) reagent:

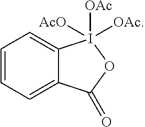

Other oxidants making it possible to oxidize a primary alcohol to aldehyde can be used, in particular, but without be limited to this, Swern's reagent (Mancuso, A. J.; Huang, S. L.; Swern, D. J. Org. Chem., 1978, 43, 2480).

Oligonucleotides are short sequences of nucleotides (a few tens of bases) which can be either DNA (oligodeoxyribonucleotides) or RNA (oligoribonucleotides).

A nucleotide is formed by one of the purine bases or pyrimidine bases, linked to a sugar residue (2-deoxyribose in the case of DNA and ribose in the case of RNA) by an N-glycosidic bond.

The chain formation of the nucleosides is ensured by phosphodiester bonds which link the C5 carbon atom of one monosaccharide unit to the C3 carbon atom of the following one.

By the expression “modified oligonucleotides”, is therefore meant oligonucleotides in which the phosphate groups, the base and/or the sugar have been modified, i.e. not having the same structure as when they are of natural origin, for example a sugar in α- instead of β-anomeric form, or a base or a sugar on which a chemical substitution has been carried out, without however being limited by these examples.

In a preferred embodiment, the invention relates to the use of a modified or non-modified oligonucleotide, with an oxidant as defined above, and:
comprising a 5′-OH end, and
containing (n+1) nucleotides,
in order to obtain respectively a modified or non-modified oligonucleotide,
comprising a 5′-phosphate monoester or 5′-thiophosphate monoester end, and
containing n nucleotides.

The reaction is diagrammatically represented below:

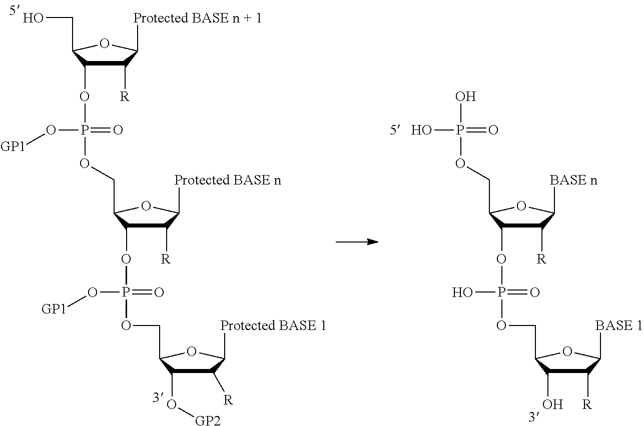

GP1 represents a protective group of the Oxidant and GP2 a protective group of the alcohol function.

The reaction therefore consists of oxidizing the 5′ alcohol of the nucleotide n+1 to aldehyde, which then undergoes β-elimination which leads to the elimination of the nucleic base n+1 in order to obtain the oligonucleotide comprising n nucleotides.

According to another embodiment, the use of a modified or non-modified oligonucleotide comprising a 5′-OH end, with an oxidant as defined above is carried out in the context of a solid phase synthesis.

The expression “solid phase synthesis” means that the synthesis is carried out in solid phase on a support constituted by a synthetic polymer, in particular, but without being limited to, a “controlled pore glass (CPG)”, i.e. a glass matrix with pores of controlled size, or polystyrene beads.

The support is situated at the alcohol in the 3′ position of the nucleotide 1 (GP 2 above).

The oxidation takes place on completion of the solid phase synthesis of the protected oligonucleotide (ODN). The ODN obtained is subjected to a first mild basic treatment, using for example, but without be limited to, a base such as potassium carbonate or methylamine, triethylamine, diisopropyl amine and the tertiary amines in general in order to promote β-elimination, followed by treatment with concentrated ammonium hydroxide.

According to a preferred embodiment, the invention comprises the use of a modified or non-modified oligonucleotide comprising a 5′-OH end, with an oxidant, as defined above, in order to carry out the synthesis of a compound of formula (I) below:

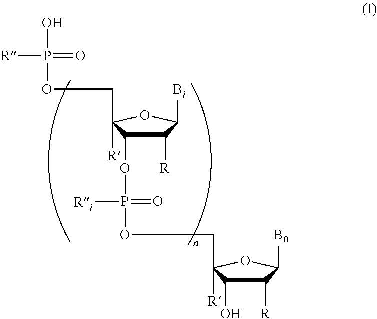

in which:
n is an integer comprised between 1 and 100, in particular 1 and 50, and in particular 5 and 40,
and i is an integer from 1 to n,
B₀ and Bᵢ represent independently of each other a nitrogenous base, in particular:
   a purine derivative, in particular adenine, guanine, 8-hydroxy guanine, 8-hydroxy-adenine, 8-amino-guanine, 8-methoxy-guanine, 8-bromo-guanine, 8-bromo-adenine, inosine, or
   a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine, but without being limited to these.
R represents:
   hydrogen, or
   a hydroxyl group, or
   a linear or branched —O-alkyl group comprising 1 to 4 carbon atoms, or
   an —O—(CH₂)ₘ—NH₂ group, in which m represents an integer comprised between 2 and 10, or
   an oxygen atom,
R′ represents:
   hydrogen, or
   a —(CH₂)ₚ— group in which p varies from 1 to 4,
such that when R represents an oxygen atom, R′ represents a —(CH₂)ₚ— group, and when R′ represents a —(CH₂)ₚ— group, R represents an oxygen atom, the R and R′ groups being bridged and then forming an ether ring, and
R″ and Rᵢ″ represent independently a hydroxyl (OH) or mercapto (SH) group.
   The oligonucleotides thus synthesized are modified or non-modified oligonucleotides of DNA or of RNA, comprising a 5′-phosphate monoester or 5′-thiophosphate monoester end.

According to an even more preferred embodiment, the invention comprises the use of a modified or non-modified oligonucleotide comprising a 5′-OH end, with an oxidant, as defined above, for carrying out the synthesis of a compound of formula (II) below:

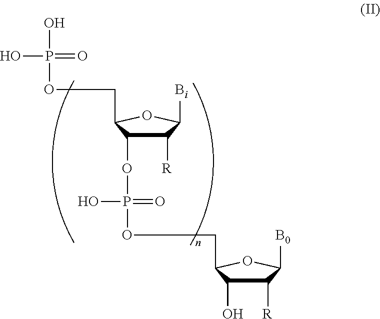

in which:
n is an integer comprised between 1 and 100, in particular 1 and 50, and in particular 5 and 40,
i is an integer comprised between 1 and n,
B₀ and Bᵢ represent independently of each other adenine, guanine, thymine, cytosine, or uracil,
R represents hydrogen or a hydroxyl group.

The oligonucleotides thus synthesized are non-modified oligonucleotides of DNA or RNA comprising a 5′-phosphate monoester end.

According to an even more preferred embodiment, the invention comprises the use of a modified or non-modified oligonucleotide comprising a 5′-OH end, with an oxidant, as defined above, for carrying out the synthesis of a compound of formula (III) below:

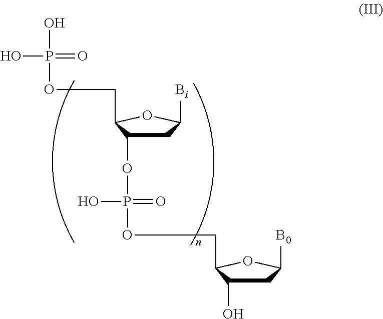

in which:
   n is an integer comprised between 1 and 100, in particular 1 and 50, and in particular 5 and 40,
   i is an integer comprised between 1 and n,
   B₀ and Bᵢ represent independently of each other adenine, guanine, thymine, or cytosine.

The oligonucleotides thus synthesized are non-modified oligonucleotides of DNA comprising a 5′-phosphate monoester end.

In a preferred embodiment, the invention comprises the use of a modified or non-modified oligonucleotide comprising a 5′-OH end, with an oxidant, as defined above, for carrying out the synthesis of a compound of formula (IV) below:

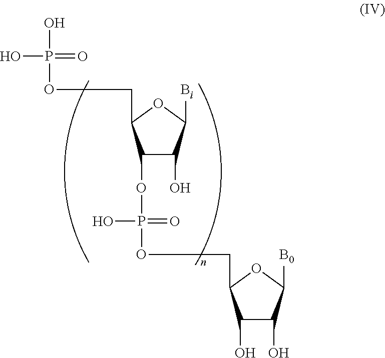

in which:
   n is an integer comprised between 1 and 100, in particular 1 and 50, and in particular 5 and 40,
   i is an integer comprised between 1 and n,
   B₀ and Bᵢ represent independently of each other adenine, guanine, cytosine, or uracil.

The oligonucleotides thus synthesized are non-modified oligonucleotides of RNA, comprising a 5′-phosphate monoester end.

In an even more preferred embodiment, the invention comprises the use of a non-modified oligonucleotide comprising a 5′-SH end, with an oxidant, as defined above, for carrying out the synthesis of a compound of formula (V′) below:

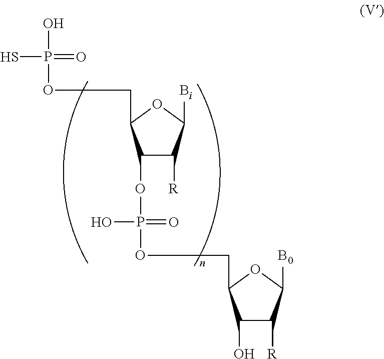

in which:
   n is an integer comprised between 1 and 100, in particular 1 and 50, and in particular 5 and 40,
   i is an integer comprised between 1 and n,
   B₀ and Bᵢ represent independently of each other adenine, guanine, thymine, cytosine, or uracil,
   R represents hydrogen or a hydroxyl group.

The oligonucleotides thus synthesized are non-modified oligonucleotides of DNA or RNA comprising a 5′-thiophosphate monoester end.

In an even more preferred embodiment, the invention comprises the use of a modified or non-modified oligonucleotide comprising a 5′-OH end, with an oxidant, as defined above, for carrying out the synthesis of a compound of formula (V) below:

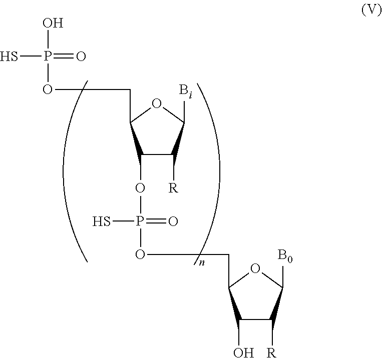

in which:
   n is an integer comprised between 1 and 100, in particular 1 and 50, and in particular 5 and 40,
   i is an integer comprised between 1 and n,
   B₀ and Bᵢ represent independently of each other a nitrogenous base, in particular:
      a purine derivative, in particular adenine, guanine, 8-hydroxy guanine, inosine, or
      a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
   R represents hydrogen or a hydroxyl group.

The oligonucleotides thus synthesized are modified oligonucleotides (of phosphorothioate type), of DNA or RNA, comprising a 5′-thiophosphate monoester end.

According to another embodiment, the invention comprises the use of a modified or non-modified oligonucleotide comprising a 5′-OH end, with an oxidant, as defined above, for carrying out the synthesis of a compound of formula (VI) below:

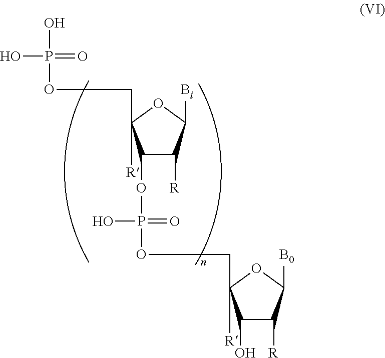

in which:
   n is an integer comprised between 1 and 100, in particular 1 and 50, and in particular 5 and 40,
   i is an integer comprised between 1 and n,
   B₀ and Bᵢ represent independently of each other a nitrogenous base, in particular:
      a purine derivative, in particular adenine, guanine, 8-hydroxy guanine, inosine, or
      a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
   R represents:
      a linear or branched —O-alkyl group comprising 1 to 4 carbon atoms, or
      an —O—(CH₂)ₘ—NH₂ group, in which m represents an integer comprised between 2 and 10, or
      an oxygen atom,
   R′ represents:
      hydrogen, or
      a —(CH₂)ₚ— group in which p varies from 1 to 4,
   such that when R represents an oxygen atom, R′ represents a —(CH₂)ₚ— group, and when R′ represents a —(CH₂)ₚ— group, R represents an oxygen atom, the R and R′ groups being bridged and then forming an ether ring.

The oligonucleotides thus synthesized are modified oligonucleotides of RNA, comprising a 5′-phosphate monoester end.

According to an even more preferred embodiment, the oxidant used for the oxidation of a modified or non-modified oligonucleotide comprising a 5′-OH end as defined above, is IBX.

The IBX can be prepared but it is also possible to use a stabilized commercial mixture containing 40% IBX and benzoic acid.

In another aspect, the invention relates to a method for preparing a compound of formula (I) below:

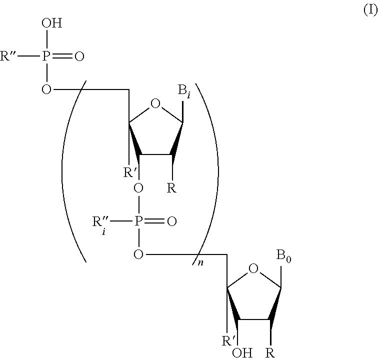

in which:
   n is an integer comprised between 1 and 100, in particular 1 and 50, and in particular 5 and 40,
   i is an integer comprised between 1 and n,
   B₀ and Bᵢ represent independently of each other a nitrogenous base, in particular:
      a purine derivative, in particular adenine, guanine, 8-hydroxy guanine, inosine, or
      a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
   R represents:
      hydrogen, or
      a hydroxyl group, or
      a linear or branched —O-alkyl group comprising 1 to 4 carbon atoms, or
      an —O—(CH₂)ₘ—NH₂ group, in which m represents an integer comprised between 2 and 10, or
      an oxygen atom,
   R′ represents:
      hydrogen, or
      a —(CH₂)ₚ— group in which p varies from 1 to 4,
   such that when R represents an oxygen atom, R′ represents a —(CH₂)ₚ— group, and when R′ represents a —(CH₂)ₚ— group, R represents an oxygen atom, the R and R′ groups being bridged and then forming an ether ring, and
   R″ and Rᵢ″ represent independently a hydroxyl or thiol group,
comprising the stage of:
reaction of an oxidant chosen from the hypervalent iodine derivatives, in particular 2-iodoxybenzoic acid (IBX) and Dess-Martin periodinane (DMP), and Swern's reagent, on a compound of formula (XI) below:

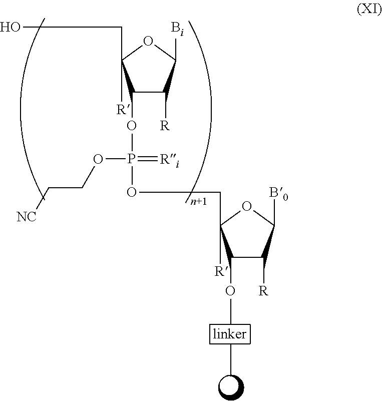

in which:
   n is an integer comprised between 1 and 100, in particular 1 and 50, and in particular 5 and 40,
   i is an integer comprised between 1 and n+1,
   B′₀ and B′ᵢ represent independently of each other a nitrogenous base, in particular:
      a purine derivative, in particular adenine, guanine, 8-hydroxy guanine, inosine, or
      a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
   said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,
   R represents:
      hydrogen, or
      a hydroxyl group protected by a protective group, in particular chosen from the acetyl (Ac), silyl such as tertbutyldimethylsilyl (TBDMS), triisopropylsilyloxymethyl (TOM) groups
      a linear or branched —O-alkyl group comprising 1 to 4 carbon atoms, or
      an —O—(CH₂)ₘ—NH₂ group, in which m represents an integer comprised between 2 and 10, and in which the amine function is protected by a protective group, in particular chosen from the carbamates such as the fluorenylmethyloxycarbonyl (Fmoc) group, the amides such as trifluoroacetamide (CO—CF3), and phthalimide,
      or
      an oxygen atom,
   R′ represents:
      hydrogen or,
      a —(CH₂)ₚ— group in which p varies from 1 to 4,
      except in the case of the last base which undergoes β-elimination, where R′=H only.
   such that when R represents an oxygen atom, R′ represents a —(CH₂)ₚ— group, and when R′ represents a —(CH₂)ₚ— group, R represents an oxygen atom, the R and R′ groups being bridged and then forming an ether ring, and
   Rᵢ″ represents independently an oxygen or sulphur atom,

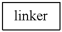

represents a linker in particular chosen from:
   a —CO—Z—CO— group, in which Z represents an alkyl group with 1 to 2 carbon atoms or a —CH₂—O-Ph-O—CH₂— group,
   a —CO—CO— group
   a group of formula (A) or (B) below:

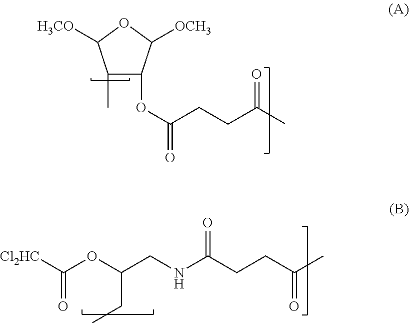

[symbol] represents a solid support, in particular chosen from CPG (Controlled Pore Glass with long chain aminoalkyl (lcaa) of different porosities) and the resins derived from polystyrene,
followed by a reaction in basic medium, in particular with ammonium hydroxide, sodium hydroxide, methylamine, potassium carbonate, or a mixture of these bases, in order to obtain said compound of formula (I).

The protective groups of the purine or pyrimidine bases are well known to a person skilled in the art and the abovementioned groups are given by way of example without any limitation.

The protective group (cyano-ethyl) of the phosphodiester bond indicated here is given only by way of example. Other protective groups of this bond well known to a person skilled in the art can also be used.

The term “linker” means that the oligonucleotide is not linked directly to the solid support but is separated from it by a spacer arm as defined above.

The linker is linked on the one hand to the support by its NH part, and on the other hand to the sugar of the nucleotide.

The CPGs are glass matrices possessing numerous pores of homogeneous size.

The expression “of different porosities” means that the CPG glass matrices can be obtained with different pore sizes and, as a result, a different porosity. The size of the pores determines the size of the oligonucleotides which can be synthesized.

The oxidation reaction is carried out in a solvent such as DMSO at a temperature comprised between 20° C. and 60° C., preferentially 40° C., for a period comprised between 15 min and 4 h, preferentially 15 min and 2 h, in particular 30 min.

The treatment in basic medium makes it possible to carry out the deprotection of the oligonucleotide from its support and to cleave the protective groups still present.

It is preferentially carried out in a 28% ammonium hydroxide solution, at a temperature comprised between 40° C. and 60° C., preferentially 55° C., for a period comprised between 14 h and 20 h, in particular 16 hours.

In a preferred embodiment, the preparation method defined above allows the preparation of a compound of formula (II) below:

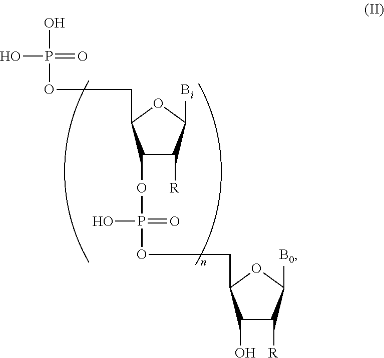

in which:
   n is an integer comprised between 1 and 100, in particular 1 and 50, and in particular 5 and 40
   i is an integer comprised between 1 and n,
   B₀ and Bᵢ represent independently of each other adenine, guanine, thymine, cytosine, or uracil,
   R represents hydrogen or a hydroxyl group
comprising the stage of:
reaction of an oxidant chosen from the hypervalent iodine derivatives, in particular 2-iodoxybenzoic acid (IBX) and Dess-Martin periodinane (DMP), and Swern's reagent, on a compound of formula (XII) below:

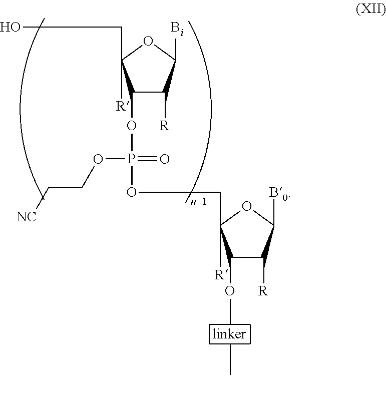

in which:
   n is an integer comprised between 1 and 100, in particular 1 and 50, and in particular 5 and 40,
   i is an integer comprised between 1 and n+1,
   B′₀ and B′ᵢ represent independently of each other a nitrogenous base chosen from adenine, guanine, thymine, cytosine, and uracil,
   said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,
   R represents hydrogen or a hydroxyl group protected by a protective group, in particular chosen from the acetyl (Ac), silyl such as tertbutyldimethylsilyl (TBDMS) and triisopropylsilyloxymethyl (TOM) groups.
   R′ represents hydrogen

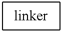

represents a linker in particular chosen from:
   a —CO—Z—CO— group, in which Z represents an alkyl group with 1 to 2 carbon atoms or a —CH₂—O-Ph-O—CH₂— group,
   a —CO—CO— group,
   a group of formula (A) or (B) below:

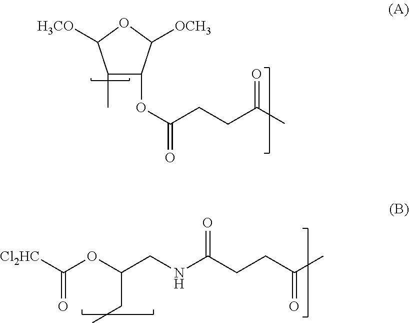

[symbol] represents a solid support in particular chosen from CPG (Controlled Pore Glass) with long chain aminoalkyl (lcaa) of different porosities and the resins derived from polystyrene,
followed by a reaction in basic medium, in particular with ammonium hydroxide, sodium hydroxide, methylamine, potassium carbonate, or a mixture of these bases, in order to obtain said compound of formula (II).

In this embodiment, non-modified oligonucleotides of DNA or RNA, comprising a 5′-phosphate monoester end are prepared.

The linker is linked on the one hand to the support by its NH part, and on the other hand to the sugar of the nucleotide.

In an even more preferred embodiment, the preparation method defined above allows the preparation of a compound of formula (III) below:

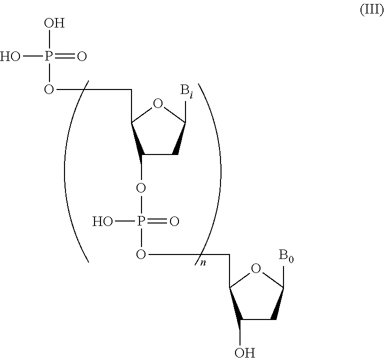

in which:
n is an integer comprised between 1 and 100, in particular 1 and 50, and in particular 5 and 40,
i is an integer comprised between 1 and n,
B₀ and Bᵢ represent independently of each other adenine, guanine, thymine, or cytosine,
comprising the stage of:
reaction of an oxidant chosen from the hypervalent iodine derivatives, in particular 2-iodoxybenzoic acid (IBX) and Dess-Martin periodinane (DMP), and Swern's reagent, on a compound of formula (XIII) below:

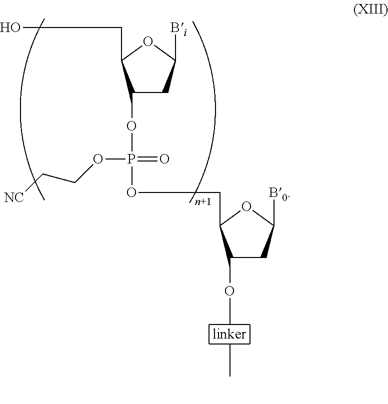

in which:
   n is an integer comprised between 1 and 100, in particular 1 and 50, and in particular 5 and 40,
   i is an integer comprised between 1 and n+1,
   B′₀ and B′ᵢ represent independently of each other a nitrogenous base chosen from adenine, guanine, thymine, and cytosine,
   said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,

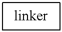

represents a linker in particular chosen from:
   a —CO—Z—CO— group, in which Z represents an alkyl group with 1 to 2 carbon atoms or a —CH₂—O-Ph-O—CH₂— group,
   a —CO—CO— group,
   a group of formula (A) or (B) below:

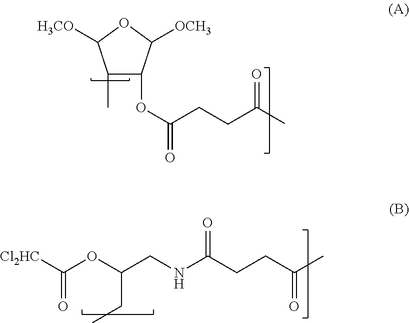

[symbol] represents a solid support in particular chosen from CPG (Controlled Pore Glass) with long chain aminoalkyl (lcaa) of different porosities and the resins derived from polystyrene,
followed by a reaction in basic medium, in particular with ammonium hydroxide, sodium hydroxide, methylamine, potassium carbonate, or a mixture of these bases, in order to obtain said compound of formula (III).

In this embodiment, non-modified oligonucleotides of DNA, comprising a 5′-phosphate monoester end are prepared.

According to another embodiment, the preparation method defined above allows the preparation of a compound of formula (IV) below:

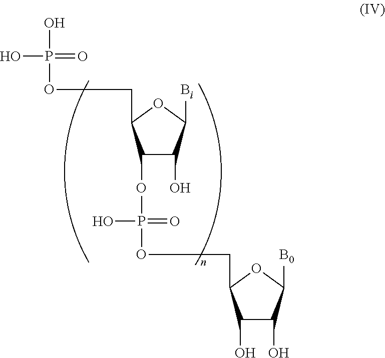

in which:
n is an integer comprised between 1 and 100, in particular 1 and 50, and in particular 5 and 40
i is an integer comprised between 1 and n,
B₀ and Bᵢ represent independently of each other adenine, guanine, cytosine, or uracil,
comprising the stage of:
reaction of an oxidant chosen from the hypervalent iodine derivatives, in particular 2-iodoxybenzoic acid (IBX) and Dess-Martin periodinane (DMP), and Swern's reagent on a compound of formula (XIV) below:

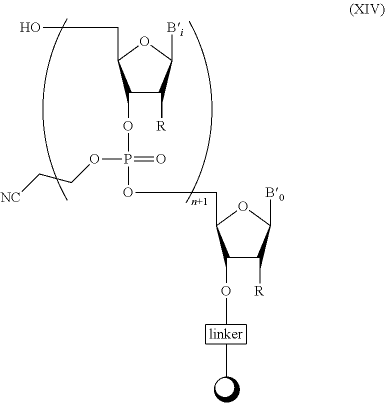

in which:
   n is an integer comprised between 1 and 100, in particular 1 and 50, and in particular 5 and 40
   i is an integer comprised between 1 and n+1,
   B′₀ and B′ᵢ represent independently of each other a nitrogenous base chosen from adenine, guanine, cytosine, and uracil,
   said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,

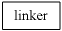

represents a linker in particular chosen from:
   a —CO—Z—CO— group, in which Z represents an alkyl group with 1 to 2 carbon atoms or a —CH₂—O-Ph-O—CH₂— group,
   a —CO—CO— group,
   a group of formula (A) or (B) below:

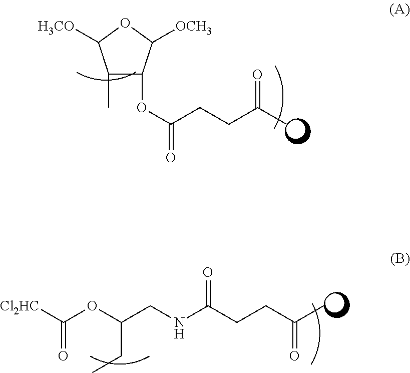

[symbol] represents a solid support in particular chosen from CPG (Controlled Pore Glass) with long chain aminoalkyl (lcaa) of different porosities and the resins derived from polystyrene,
followed by a reaction in basic medium, in particular with ammonium hydroxide, sodium hydroxide, methylamine, potassium carbonate, or a mixture of these bases, in order to obtain said compound of formula (IV).

In this embodiment, non-modified oligonucleotides of RNA, comprising a 5′-phosphate monoester end are prepared.

According to another embodiment, the preparation method defined above allows the preparation of a compound of formula (V′) below:

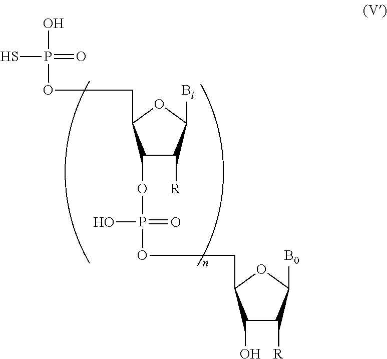

in which:
   n is an integer comprised between 1 and 100, in particular 1 and 50, and in particular 5 and 40,
   i is an integer comprised between 1 and n,
   B₀ and Bᵢ represent independently of each other adenine, guanine, thymine, cytosine, or uracil,
   R represents hydrogen or a hydroxyl group,
comprising the stage of:
reaction of an oxidant chosen from the hypervalent iodine derivatives, in particular 2-iodoxybenzoic acid (IBX) and Dess-Martin periodinane (DMP), and Swern's reagent, on a compound of formula (XXI) below:

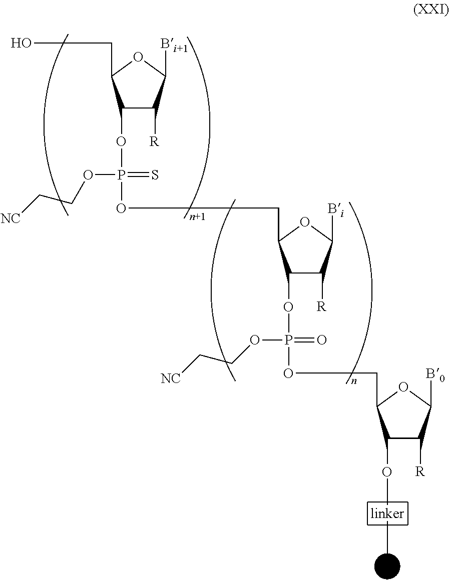

n is an integer comprised between 1 and 100, in particular 1 and 50, and in particular 5 and 40
i is an integer comprised between 1 and n+1,
B′₀, B′ᵢ and B′ᵢ₊₁ represent independently of each other a nitrogenous base chosen from adenine, guanine, cytosine, and uracil,
said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,
R represents a hydroxyl group protected by a protective group, in particular chosen from the acetyl (Ac), silyl such as tertbutyldimethylsilyl (TBDMS) and triisopropylsilyloxymethyl (TOM) groups.

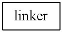

represents a linker in particular chosen from:
   a —CO—Z—CO— group, in which Z represents an alkyl group with 1 to 2 carbon atoms or a —CH₂—O-Ph-O—CH₂— group,
   a —CO—CO— group,
   a group of formula (A) or (B) below:

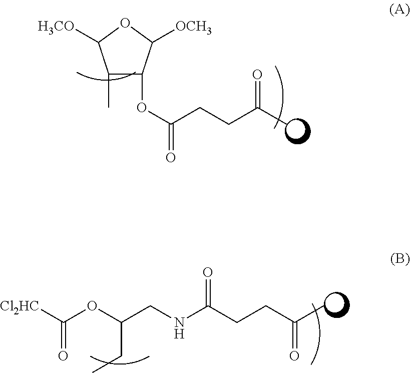

[symbol] represents a solid support in particular chosen from CPG (Controlled Pore Glass) with long chain aminoalkyl (lcaa) of different porosities and the resins derived from polystyrene,
followed by a reaction in basic medium, in particular with ammonium hydroxide, sodium hydroxide, methylamine, potassium carbonate, or a mixture of these bases, in order to obtain said compound of formula (V′).

The oligonucleotides thus synthesized are non-modified oligonucleotides of DNA or RNA comprising a 5′-thiophosphate monoester end.

According to another even more preferred embodiment, the preparation method defined above allows the preparation of a compound of formula (V) below:

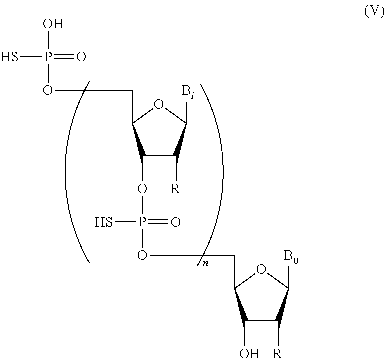

in which:
   n is an integer comprised between 1 and 100, in particular 1 and 50, and in particular 5 and 40
   i is an integer comprised between 1 and n,
   B₀ and Bᵢ represent independently of each other a nitrogenous base, in particular:
      a purine derivative, in particular adenine, guanine, 8-hydroxy guanine, inosine, or
      a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
   R represents hydrogen or a hydroxyl group,
   comprising the stage of:
   reaction of an oxidant chosen from the hypervalent iodine derivatives, in particular 2-iodoxybenzoic acid (IBX) and Dess-Martin periodinane (DMP), and Swern's reagent, on a compound of formula (XV) below:

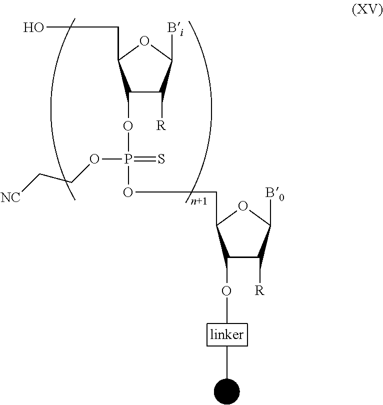

in which:
   n is an integer comprised between 1 and 100, in particular 1 and 50, and in particular 5 and 40
   i is an integer comprised between 1 and n+1,
   B′₀ and B′ᵢ represent independently of each other a nitrogenous base, in particular:
      a purine derivative, in particular adenine, guanine, 8-hydroxy guanine, inosine, or
      a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
   said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,
   R represents hydrogen or a hydroxyl group protected by a protective group, in particular chosen from the acetyl (Ac), silyl such as tertbutyldimethylsilyl (TBDMS) and triisopropylsilyloxymethyl (TOM) groups.

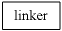

in particular chosen from:
   a —CO—Z—CO— group, in which Z represents an alkyl group with 1 to 2 carbon atoms or a —CH₂—O-Ph-O—CH₂— group,
   a —CO—CO— group,
   a group of formula (A) or (B) below:

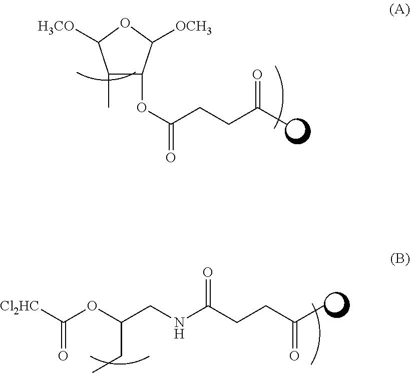

[symbol] represents a solid support in particular chosen from CPG (Controlled Pore Glass) with long chain aminoalkyl (lcaa) of different porosities and the resins derived from polystyrene,
followed by a reaction in basic medium, in particular with ammonium hydroxide, sodium hydroxide, methylamine, potassium carbonate, or a mixture of these bases, in order to obtain said compound of formula (V).

In this embodiment, modified oligonucleotides of DNA and RNA (of phosphorothioate type), and comprising a 5′-thiophosphate monoester end are prepared.

According to another even more preferred embodiment, the preparation method defined above allows the preparation of a compound of formula (VI) below:

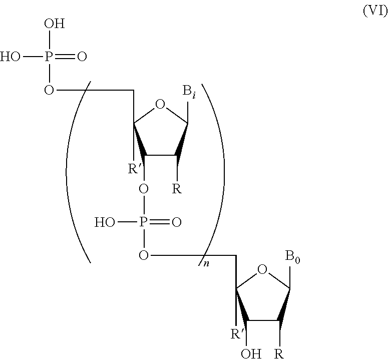

in which:
   n is an integer comprised between 1 and 100, in particular 1 and 50, and in particular 5 and 40
   i is an integer comprised between 1 and n,
   B₀ and Bᵢ represent independently of each other a nitrogenous base, in particular:
      a purine derivative, in particular adenine, guanine, 8-hydroxy guanine, inosine, or
      a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
   R represents:
      a linear or branched —O-alkyl group comprising 1 to 4 carbon atoms, or
      an —O—(CH₂)ₘ—NH₂, group in which m represents an integer comprised between 2 and 10, or
      an oxygen atom,
   R′ represents:
      hydrogen, or
      a —(CH₂)ₚ— group in which p varies from 1 to 4,
   such that when R represents an oxygen atom, R′ represents a —(CH₂)ₚ— group, and when R′ represents a —(CH₂)ₚ— group, R represents an oxygen atom, the R and R′ groups being bridged and then forming an ether ring, and
comprising the stage of:
reaction of an oxidant chosen from the hypervalent iodine derivatives, in particular 2-iodoxybenzoic acid (IBX) and Dess-Martin periodinane (DMP), and Swern's reagent, on a compound of formula (XVI) below:

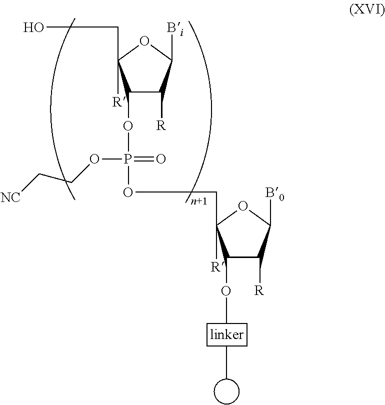

in which:
   n is an integer comprised between 1 and 100, in particular 1 and 50, and in particular 5 and 40,
   i is an integer comprised between 1 and n,
   B′₀ and B′ᵢ represent independently of each other a nitrogenous base, in particular:
      a purine derivative, in particular adenine, guanine, 8-hydroxy guanine, inosine, or
      a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
   said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,
   R represents:
      a linear or branched —O-alkyl group comprising 1 to 4 carbon atoms, or
      an —O—(CH₂)ₘ—NH₂ group, in which the amine function is protected by a protective group, in particular chosen from the carbamates such as the fluorenylmethyloxycarbonyl (Fmoc) group, amides such as trifluoroacetamide (CO—CF3), and phthalimide,
      in which m represents an integer comprised between 2 and 10, or
      an oxygen atom,
   R′ represents:
      hydrogen, or
      a —(CH₂)ₚ— group in which p varies from 1 to 4,
   such that when R represents an oxygen atom, R′ represents a —(CH₂)ₚ— group, and when R′ represents a —(CH₂)ₚ— group, R represents an oxygen atom, the R and R′ groups being bridged and then forming an ether ring, except in the case of the last base which undergoes β-elimination, where R′ represents hydrogen only.
   and,
   Rᵢ″ represents independently a hydroxyl or thiol group,

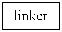

in particular chosen from:
   a —CO—Z—CO— group, in which Z represents an alkyl group with 1 to 2 carbon atoms or a —CH₂—O-Ph-O—CH₂— group,
   a —CO—CO— group,
   a group of formula (A) or (B) below:

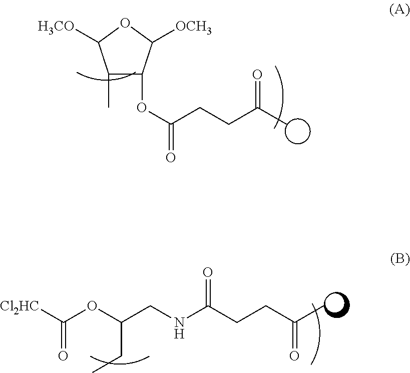

[symbol] represents a solid support in particular chosen from CPG (Controlled Pore Glass) with long chain aminoalkyl (lcaa) of different porosities and the resins derived from polystyrene,
followed by a reaction in basic medium, in particular with ammonium hydroxide, sodium hydroxide, methylamine, potassium carbonate, or a mixture of these bases, in order to obtain said compound of formula (VI).

In this embodiment, modified oligonucleotides of RNA, comprising a 5′-phosphate monoester end are prepared.

According to another aspect, the invention relates to a method for preparing a compound of formula (XX) below:

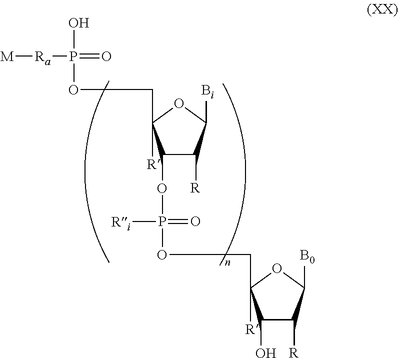

in which:
   n is an integer comprised between 1 and 100, in particular 1 and 50, and in particular 5 and 40,
   i is an integer comprised between 1 and n,
   B₀ and Bᵢ represent independently of each other a nitrogenous base, in particular:
      a purine derivative, in particular adenine, guanine, 8-hydroxy guanine, inosine, or
      a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
   R represents:
      hydrogen, or
      a hydroxyl group, or
      a linear or branched —O-alkyl group comprising 1 to 4 carbon atoms, or
      an oxygen atom,
   Rᵢ″ represents independently a hydroxyl or thiol group,
   Ra represents a sulphur or oxygen atom,
   M represents a fluorescent probe, a peptide, an oligonucleotide or an intercalating agent,
comprising the stage of:
reaction of an oxidant chosen from the hypervalent iodine derivatives, in particular 2-iodoxybenzoic acid (IBX) and Dess-Martin periodinane (DMP), and Swern's reagent, on a compound of formula (XI) below:

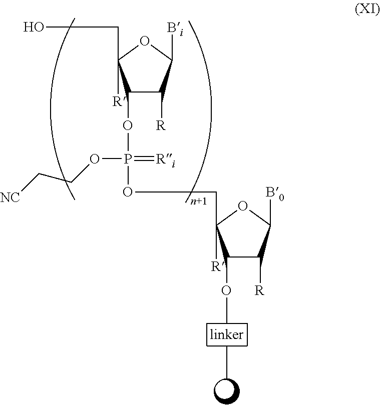

in which:
   n is an integer comprised between 1 and 100, in particular 1 and 50, and in particular 5 and 40,
   i is an integer comprised between 1 and n+1,
   B′₀ and B′ᵢ represent independently of each other a nitrogenous base, in particular:
      a purine derivative, in particular adenine, guanine, 8-hydroxy guanine, inosine, or
      a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
   said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,
   R represents:
      hydrogen, or
      a hydroxyl group, protected by a protective group, in particular chosen from the acetyl (Ac), silyl such as tertbutyldimethylsilyl (TBDMS) and triisopropylsilyloxymethyl (TOM) groups or
      a linear or branched —O-alkyl group comprising 1 to 4 carbon atoms, or
      an oxygen atom,
   R′ represents:
      hydrogen, or
      a —(CH₂)ₚ— group in which p varies from 1 to 4,
   such that when R represents an oxygen atom, R′ represents a —(CH₂)ₚ— group, and when R′ represents a —(CH₂)ₚ— group, R represents an oxygen atom, the R and R′ groups being bridged and then forming an ether ring,
   except in the case of the last base which undergoes β-elimination, where R′ represents hydrogen only.
   Rᵢ″ represents independently an oxygen or sulphur atom,

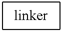

represents a linker in particular chosen from:
   a —CO—Z—CO— group, in which Z represents an alkyl group with 1 to 2 carbon atoms or a —CH₂—O-Ph-O—CH₂— group,
   a —CO—CO— group,
   a group of formula (A) or (B) below:

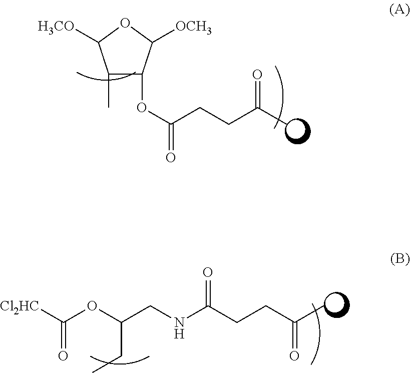

[symbol] represents a solid support, in particular chosen from CPG (Controlled Pore Glass) with long chain aminoalkyl (lcaa) of different porosities and the resins derived from polystyrene,
followed by a reaction with a molecule M-GP, in which M represents a fluorescent probe, a peptide, an oligonucleotide or an intercalating agent, and GP represents a leaving group, in particular chosen from chlorine, bromine, iodine, the tosylate, mesylate groups, when Ra is a sulphur atom,
or with a molecule M-OH or M-NH2 after activation of the 5′-phosphate when Ra is an oxygen atom.
and by a reaction in basic medium, in particular with ammonium hydroxide, sodium hydroxide, methylamine, potassium carbonate, or a mixture of these bases,
said reactions with a molecule M-GP, M-OH or M-NH₂ and in basic medium being able to be carried out successively or in a single “one-pot” stage,
in order to obtain said compound of formula (XX).

By “fluorescent probe”, is meant molecules having luminescence properties such as cyanine, fluorescein, Texas Red without being limited to these.

By “oligonucleotide”, is meant an oligonucleotide synthesized by a method as above or by a method well known to a person skilled in the art and allowing the synthesis of longer oligonucleotides, in particular by convergent synthesis by fragments.

By “intercalating agent”, is meant molecules of polyaromatic type having DNA intercalating properties such as acridine, quinacridine, without being limited to these.

The treatment in basic medium as above makes it possible to carry out the deprotection of the oligonucleotide from its support and to cleave the protective groups also present.

According to a preferred embodiment, the oxidant used in the above methods is IBX.

### EXPERIMENTAL PART

### 1) General Diagram of Synthesis of Oligonucleotides Comprising a 5′-Phosphate or 5′-Thiophosphate Monoester Bond

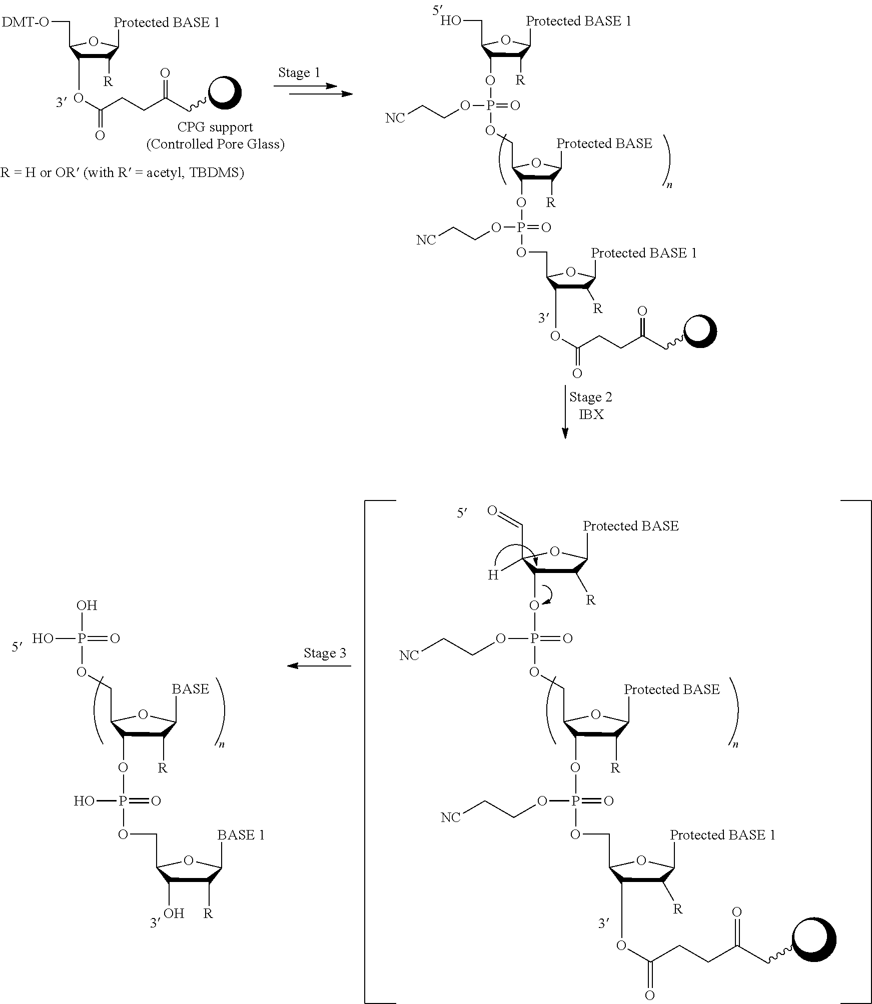

Stage 1 corresponds to the complete cycle of oligonucleotide synthesis by the phosphoramidite route and consists of deprotection (detritylation) with trichloroacetic or dichloroacetic acid, followed by coupling with the following base (n+1) in the form of phosphoramidite, oxidation of phosphorus III to phosphorus (V) by an aqueous solution of iodine, “capping” (protection of the alcohols in the 5′ position which have not reacted) by acetylation.

Stage 2 corresponds to the stage of oxidation with the hypervalent iodine derivative.

Stage 3 corresponds to deprotection with ammonium hydroxide.

This diagram shows the molecules in which R′=H.

This same diagram is also valid in the case where R′=a —(CH₂)ₚ— group in which p varies from 1 to 4, provided that for the last base, R′=H.

The formation of the 5′-phosphate is probably due to a β-elimination mechanism by removal of the acid proton in a position from the aldehyde generated by the IBX oxidation.

Once the elimination has been carried out, it is then possible to react a molecule M-PG on the non-isolated intermediate in order to obtain the compounds defined above.

### 2) Protocol for Conventional Oligonucleotides (in DNA or RNA Series)

The oligonucleotide is prepared by the standard solid phase synthesis route (phosphoramidite route). On completion of the synthesis, the oligonucleotide is attached by its 3′ end to the solid support in completely protected form. Thus, the exocyclic amine groups of the nucleobases are protected in the form of benzoyl (for dA and dC) or isobutyryl (for dG) or other commercial protective groups, the internucleosidic phosphodiester in the form of cyanoethyl and the alcohol in the 3′ position in the form of ester with the support.

In the RNA series, the alcohol function in the 2′ position is protected in the form of TBDMS or acetyl or other protective groups described for this protection. This support is then put into a solution of IBX in DMSO (commercial 100 mM solution) and heated at 60° for 16 hours. The purpose of this stage is to oxidize the primary alcohol function in the 5′ position to aldehyde.

The solid support is then recovered and placed in a 28% ammonium hydroxide solution and heated at 55° C. for 16 hours. This treatment is commonly used in the conventional synthesis of oligonucleotides and corresponds to the stage of cleavage of the oligonucleotide from the support and deprotection of the amines of the nucleobases and the phosphodiester bond. The oligonucleotide is then purified according to the standard methods (high performance liquid chromatography or gel electrophoresis).

### 3) Protocol for Modified Oligonucleotides

The synthesis protocol is the same as that described previously for the natural oligonucleotides. The modification is incorporated according to the protocol described by the constructor.

For example, the phosphorothioate bonds can replace the phosphodiester bonds using Beaucage reagent during the oxidation stage, the OH function in the 2′ position may be in the alkoxy form or attached via a methylene in the 4′ position (LNA).

1-20. (canceled)

21. A method for the synthesis of modified or non-modified oligonucleotides, comprising reacting a modified or non-modified oligonucleotides comprising a 5′-phosphate monoester or 5′-thiophosphate monoester end with at least one oxidant chosen from:
the hypervalent iodine derivatives, in particular 2-iodoxybenzoic acid (IBX) and Dess-Martin periodinane (DMP), and
Swern's reagent.

22. The method according to claim 21, wherein the modified or non-modified oligonucleotide,
comprises a 5′-OH end, and
contains (n+1) nucleotides,
in order to obtain respectively a modified or non-modified oligonucleotide,
comprising a 5′-phosphate monoester or 5′-thiophosphate monoester end, and
containing n nucleotides.

23. The method according to claim 21 in which the synthesis is a solid phase synthesis.

24. The method according to claim 21, wherein the method is for the synthesis of a compound of formula (I) below: 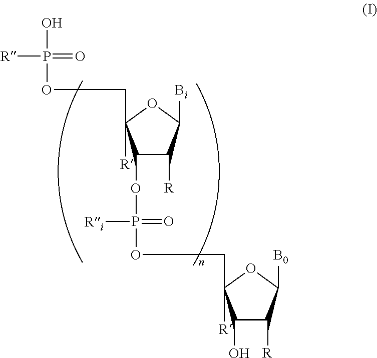
in which:
n is an integer comprised between 1 and 100, in particular 1 and 50, and in particular 5 and 40,
i is an integer comprised between 1 and n,
B₀ and Bᵢ represent independently of each other a nitrogenous base, in particular:
a purine derivative, in particular adenine, guanine, 8-hydroxy guanine, 8-hydroxy-adenine, 8-amino-guanine, 8-methoxy-guanine, 8-bromo-guanine, 8-bromo-adenine, inosine, or
a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
R represents:
hydrogen, or
a hydroxyl group, or
a linear or branched —O-alkyl group comprising 1 to 4 carbon atoms, or
an —O—(CH₂)ₘ—NH₂ group, in which m represents an integer comprised between 2 and 10, or
an oxygen atom,
R′ represents:
hydrogen, or
a —(CH₂)ₚ— group in which p varies from 1 to 4,
such that when R represents an oxygen atom, R′ represents a —(CH₂)ₚ— group, and when R′ represents a —(CH₂)ₚ— group, R represents an oxygen atom, the R and R′ groups being bridged and then forming an ether ring, and
R″ and Rᵢ″ represent independently a hydroxyl (OH) or mercapto (SH) group.

25. The method according to claim 24, wherein the method is for the synthesis of a compound of formula (II) below: 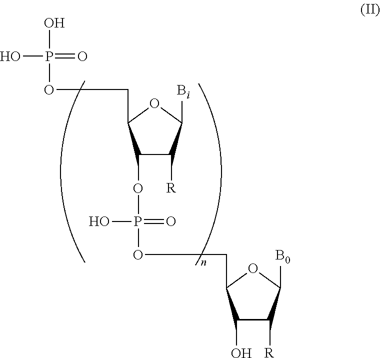
in which:
n is an integer comprised between 1 and 100, in particular 1 and 50, and in particular 5 and 40,
i is an integer comprised between 1 and n,
B₀ and Bᵢ represent independently of each other adenine, guanine, thymine, cytosine, or uracil, and
R represents hydrogen or a hydroxyl group.

26. The method according to claim 24, wherein the method is for the synthesis of a compound of formula (III) below: 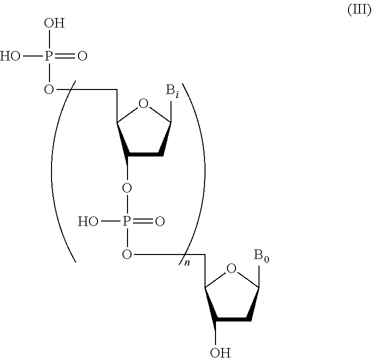
in which:
n is an integer comprised between 1 and 100, in particular 1 and 50, and in particular 5 and 40,
i is an integer comprised between 1 and n, and
B₀ and Bᵢ represent independently of each other adenine, guanine, thymine, or cytosine.

27. The method according to claim 24, wherein the method is for the synthesis of a compound of formula (IV) below: 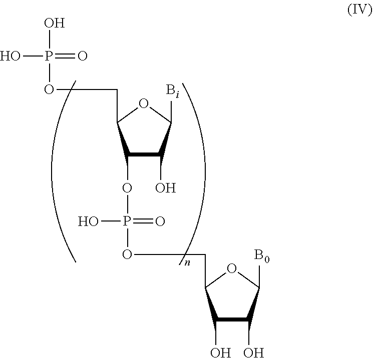
in which:
n is an integer comprised between 1 and 100, in particular 1 and 50, and in particular 5 and 40,
i is an integer comprised between 1 and n, and
B₀ and Bᵢ represent independently of each other adenine, guanine, cytosine, or uracil.

28. The method according to claim 24 for the synthesis of a compound of formula (V′) below: 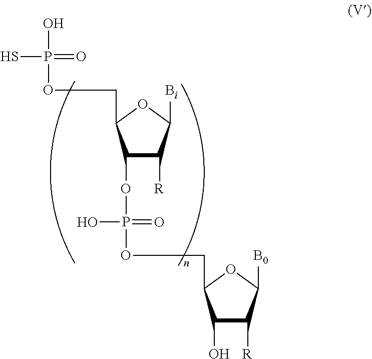
in which:
n is an integer comprised between 1 and 100, in particular 1 and 50, and in particular 5 and 40,
i is an integer comprised between 1 and n,
B₀ and Bᵢ represent independently of each other adenine, guanine, cytosine, or uracil, and
R represents hydrogen or a hydroxyl group.

29. The method according to claim 24, wherein the method is for the synthesis of a compound of formula (V) below: 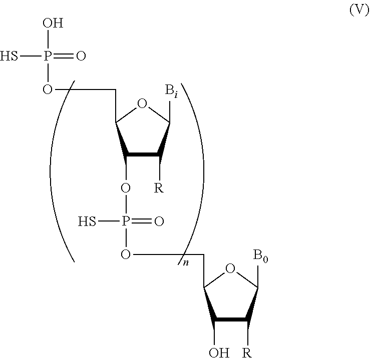
in which:
n is an integer comprised between 1 and 100, in particular 1 and 50, and in particular 5 and 40,
i is an integer comprised between 1 and n,
B₀ and Bᵢ represent independently of each other a nitrogenous base, in particular:
a purine derivative, in particular adenine, guanine, 8-hydroxy guanine, inosine, or
a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine, and
R represents hydrogen or a hydroxyl group.

30. The method according to claim 24, wherein the method is for the synthesis of a compound of formula (VI) below: 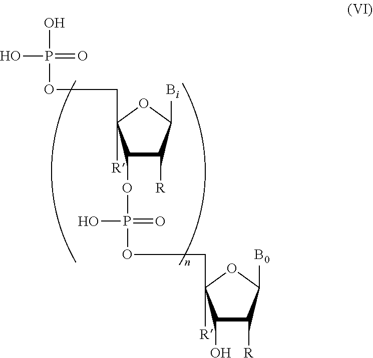
in which:
n is an integer comprised between 1 and 100, in particular 1 and 50, and in particular 5 and 40,
i is an integer comprised between 1 and n,
B₀ and Bᵢ represent independently of each other a nitrogenous base, in particular:
a purine derivative, in particular adenine, guanine, 8-hydroxy guanine, inosine, or
a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
R represents:
a linear or branched —O-alkyl group comprising 1 to 4 carbon atoms, or
an —O—(CH₂)ₘ—NH₂ group, in which m represents an integer comprised between 2 and 10, or
an oxygen atom,
R′ represents:
hydrogen, or
a —(CH₂)ₚ— group in which p varies from 1 to 4,
such that when R represents an oxygen atom, R′ represents a —(CH₂)ₚ— group, and when R′ represents a —(CH₂)ₚ— group, R represents an oxygen atom, the R and R′ groups being bridged and then forming an ether ring.

31. The method according to claim 21, wherein the oxidant is IBX.

32. A method for preparing a compound of formula (I) below: 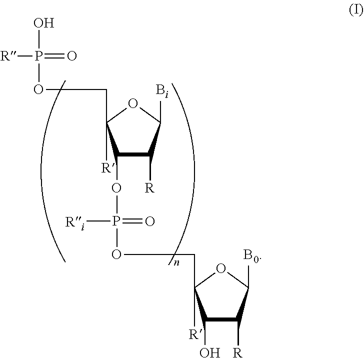
in which:
n is an integer comprised between 1 and 100, in particular 1 and 50, and in particular 5 and 40,
i is an integer comprised between 1 and n,
B₀ and Bᵢ represent independently of each other a nitrogenous base, in particular:
a purine derivative, in particular adenine, guanine, 8-hydroxy guanine, inosine, or
a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
R represents:
hydrogen, or
a hydroxyl group, or
a linear or branched —O-alkyl group comprising 1 to 4 carbon atoms, or
an —O—(CH₂)ₘ—NH₂ group, in which m represents an integer comprised between 2 and 10, or
an oxygen atom,
R′ represents:
hydrogen, or
a —(CH₂)ₚ— group in which p varies from 1 to 4,
such that when R represents an oxygen atom, R′ represents a —(CH₂)ₚ— group, and when R′ represents a —(CH₂)ₚ— group, R represents an oxygen atom, the R and R′ groups being bridged and then forming an ether ring, and
R″ and Rᵢ″ represent independently a hydroxyl or thiol group,
comprising the stage of:
reaction of an oxidant chosen from the hypervalent iodine derivatives, in particular 2-iodoxybenzoic acid (IBX) and Dess-Martin periodinane (DMP), and Swern's reagent, on a compound of formula (XI) below: 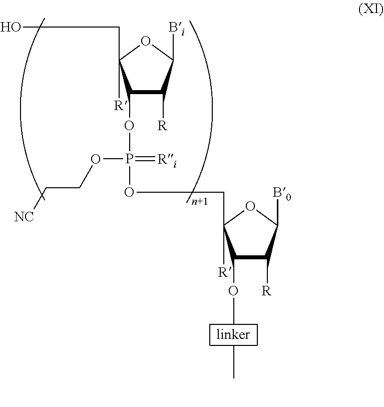
in which:
n is an integer comprised between 1 and 100, in particular 1 and 50, and in particular 5 and 40,
i is an integer comprised between 1 and n+1,
B′₀ and B′ᵢ represent independently of each other a nitrogenous base, in particular:
a purine derivative, in particular adenine, guanine, 8-hydroxy guanine, inosine, or
a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,
R represents:
hydrogen, or
a hydroxyl group protected by a protective group, in particular chosen from the acetyl (Ac), silyl such as tertbutyldimethylsilyl (TBDMS), triisopropylsilyloxymethyl (TOM) groups,
a linear or branched —O-alkyl group comprising 1 to 4 carbon atoms, or
an —O—(CH₂)ₘ—NH₂ group, in which m represents an integer comprised between 2 and 10, and in which the amine function is protected by a protective group, in particular chosen from the carbamates such as the fluorenylmethyloxycarbonyl (Fmoc) group, the amides such as trifluoroacetamide (CO—CF3), and phthalimide,
or
an oxygen atom,
R′ represents:
hydrogen or,
a —(CH₂)ₚ— group in which p varies from 1 to 4,
except in the case of the last base, where R′ represents hydrogen,
such that when R represents an oxygen atom, R′ represents a —(CH₂)ₚ— group, and when R′ represents a —(CH₂)ₚ— group, R represents an oxygen atom, the R and R′ groups being bridged and then forming an ether ring, and
Rᵢ″ represents independently an oxygen or sulphur atom, 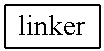
represents a linker in particular chosen from:
a —CO—Z—CO— group, in which Z represents an alkyl group with 1 to 2 carbon atoms or a —CH₂—O-Ph-O—CH₂— group,
a —CO—CO— group,
a group of formula (A) or (B) below: 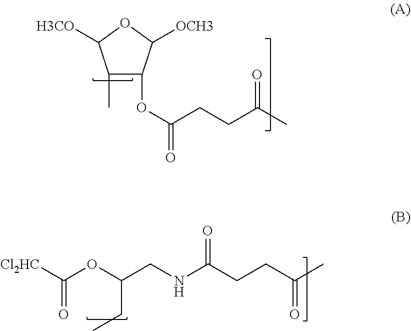 [symbol]
represents a solid support, in particular chosen from CPG (Controlled Pore Glass) with long chain aminoalkyl (lcaa) of different porosities and the resins derived from polystyrene,
followed by a reaction in basic medium, in particular with ammonium hydroxide, sodium hydroxide, methylamine, potassium carbonate, or a mixture of these bases, in order to obtain said compound of formula (I).

33. The method according to claim 32, wherein the compound prepared is further defined according to formula (II) below: 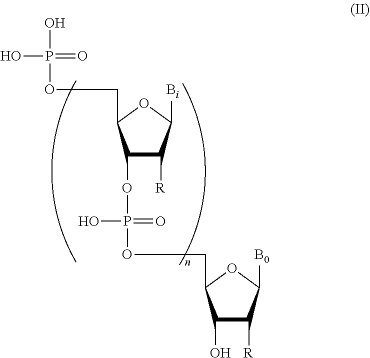
in which:
n is an integer comprised between 1 and 100, in particular 1 and 50, and in particular 5 and 40
i is an integer comprised between 1 and n,
B₀ and Bᵢ represent independently of each other adenine, guanine, thymine, cytosine, or uracil,
R represents hydrogen or a hydroxyl group, and
the stage of reaction involves the oxidant and a compound on a compound further defined according to formula (XII) below: 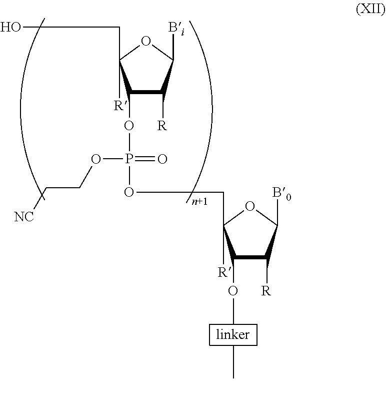
in which:
n is an integer comprised between 1 and 100, in particular 1 and 50, and in particular 5 and 40,
i is an integer comprised between 1 and n+1,
B′₀ and B′ᵢ represent independently of each other a nitrogenous base chosen from adenine, guanine, thymine, cytosine, and uracil,
said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,
R represents hydrogen or a hydroxyl group,
R′ represents hydrogen 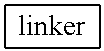
represents a linker in particular chosen from:
a —CO—Z—CO— group, in which Z represents an alkyl group with 1 to 2 carbon atoms or a —CH₂—O-Ph-O—CH₂— group,
a —CO—CO— group,
a group of formula (A) or (B) below: 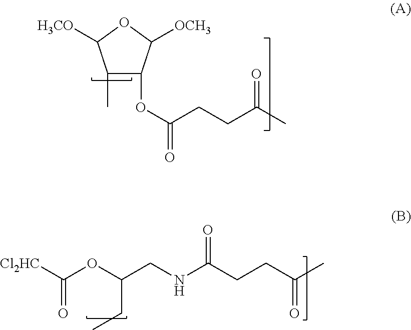 [symbol]
represents a solid support in particular chosen from CPG (Controlled Pore Glass) with long chain aminoalkyl (lcaa) of different porosities and the resins derived from polystyrene, and
the reaction is followed by a reaction in basic medium, in particular with ammonium hydroxide, sodium hydroxide, methylamine, potassium carbonate, or a mixture of these bases, in order to obtain said compound of formula (II).

34. The method according to claim 32, wherein the compound prepared is further defined according to formula (III) below: 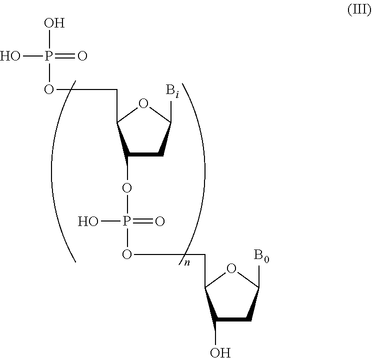
in which:
n is an integer comprised between 1 and 100, in particular 1 and 50, and in particular 5 and 40,
i is an integer comprised between 1 and n,
B₀ and Bᵢ represent independently of each other adenine, guanine, thymine, or cytosine, and
the stage of reaction involves the oxidant and a compound of formula (XIII) below: 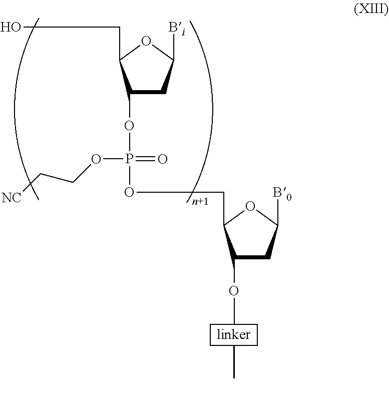
in which:
n is an integer comprised between 1 and 100, in particular 1 and 50, and in particular 5 and 40,
i is an integer comprised between 1 and n+1,
B′₀ and B′ᵢ represent independently of each other a nitrogenous base chosen from adenine, guanine, thymine, and cytosine,
said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups, 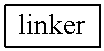
represents a linker in particular chosen from:
a —CO—Z—CO— group, in which Z represents an alkyl group with 1 to 2 carbon atoms or a —CH₂—O-Ph-O—CH₂— group,
a —CO—CO— group,
a group of formula (A) or (B) below: 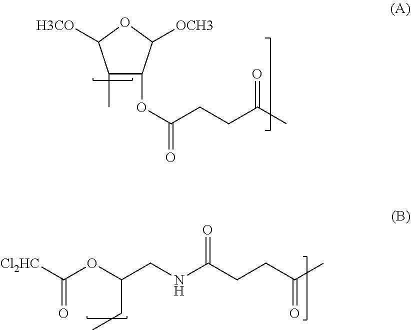 [symbol]
represents a solid support in particular chosen from CPG (Controlled Pore Glass) with long chain aminoalkyl (lcaa) of different porosities and the resins derived from polystyrene, and
the reaction is followed by a reaction in basic medium, in particular with ammonium hydroxide, sodium hydroxide, methylamine, potassium carbonate, or a mixture of these bases, in order to obtain said compound of formula (III).

35. The method according to claim 32, wherein the compound prepared is further defined according to formula (IV) below: 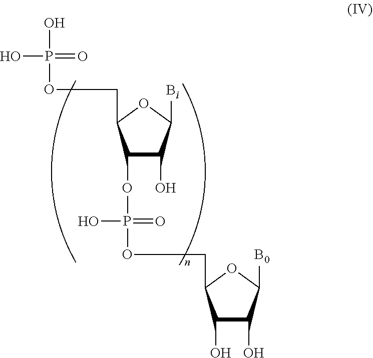
in which:
n is an integer comprised between 1 and 100, in particular 1 and 50, and in particular 5 and 40
i is an integer comprised between 1 and n,
B₀ and Bᵢ represent independently of each other adenine, guanine, cytosine, or uracil, and
the stage of reaction involves the oxidant and a compound of formula (XIV) below: 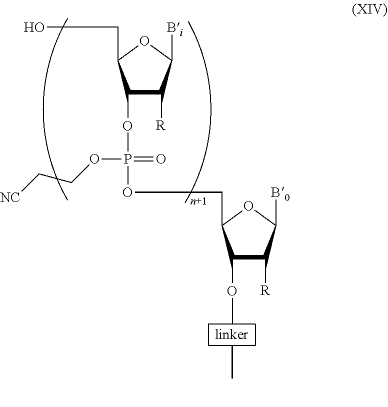
in which:
n is an integer comprised between 1 and 100, in particular 1 and 50, and in particular 5 and 40
i is an integer comprised between 1 and n+1,
B′₀ and B′ᵢ represent independently of each other a nitrogenous base chosen from adenine, guanine, cytosine, and uracil,
said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups, 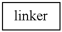
represents a linker in particular chosen from:
a —CO—Z—CO— group, in which Z represents an alkyl group with 1 to 2 carbon atoms or a —CH₂—O-Ph-O—CH₂— group,
a —CO—CO— group,
a group of formula (A) or (B) below: 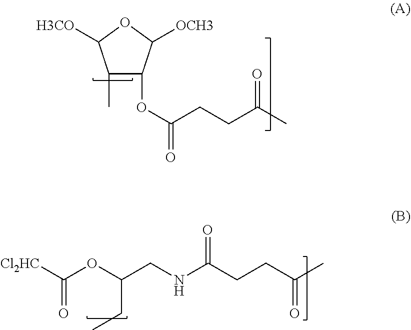 [symbol]
represents a solid support in particular chosen from CPG (Controlled Pore Glass) with long chain aminoalkyl (lcaa) of different porosities and the resins derived from polystyrene, and
the reaction is followed by a reaction in basic medium, in particular with ammonium hydroxide, sodium hydroxide, methylamine, potassium carbonate, or a mixture of these bases, in order to obtain said compound of formula (IV).

36. The method according to claim 32, wherein the compound prepared is further defined according to formula (V′) below: 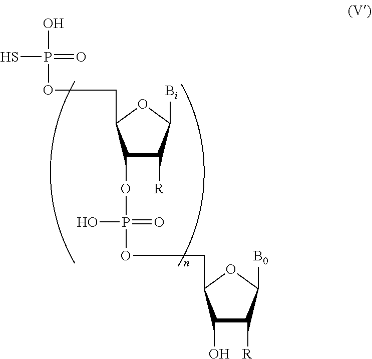
in which:
n is an integer comprised between 1 and 100, in particular 1 and 50, and in particular 5 and 40,
i is an integer comprised between 1 and n,
B₀ and Bᵢ represent independently of each other adenine, guanine, thymine, cytosine, or uracil,
R represents hydrogen or a hydroxyl group,
the stage of reaction involves the oxidant and a compound of formula (XXI) below: 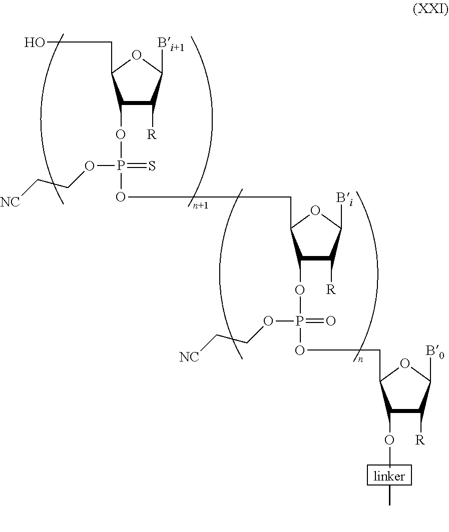
in which:
n is an integer comprised between 1 and 100, in particular 1 and 50, and in particular 5 and 40
i is an integer comprised between 1 and n+1,
B′₀, B′ᵢ and B′ᵢ₊₁ represent independently of each other a nitrogenous base chosen from adenine, guanine, cytosine, and uracil,
said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,
R represents a hydroxyl group protected by a protective group, in particular chosen from the acetyl (Ac), silyl such as tertbutyldimethylsilyl (TBDMS) and triisopropylsilyloxymethyl (TOM) groups. 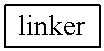
represents a linker in particular chosen from:
a —CO—Z—CO— group, in which Z represents an alkyl group with 1 to 2 carbon atoms or a —CH₂—O-Ph-O—CH₂— group,
a —CO—CO— group,
a group of formula (A) or (B) below: 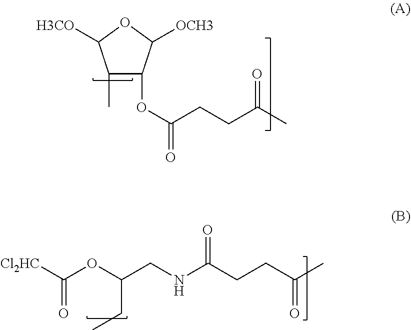 [symbol]
represents a solid support in particular chosen from CPG (Controlled Pore Glass) with long chain aminoalkyl (lcaa) of different porosities and the resins derived from polystyrene, and
the reaction is followed by a reaction in basic medium, in particular with ammonium hydroxide, sodium hydroxide, methylamine, potassium carbonate, or a mixture of these bases, in order to obtain said compound of formula (V′).

37. The method according to claim 32, wherein the compound prepared is further defined according to formula (V) below: 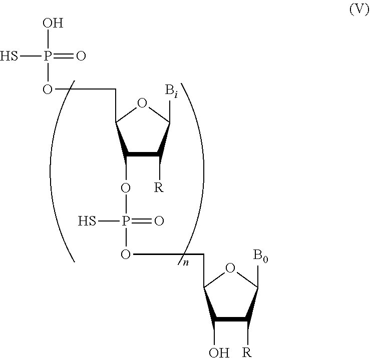
in which:
n is an integer comprised between 1 and 100, in particular 1 and 50, and in particular 5 and 40
i is an integer comprised between 1 and n,
B₀ and Bᵢ represent independently of each other a nitrogenous base, in particular:
a purine derivative, in particular adenine, guanine, 8-hydroxy guanine, inosine, or
a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
R represents hydrogen or a hydroxyl group, and
the stage of reaction involves the oxidant and a compound of formula (XV) below: 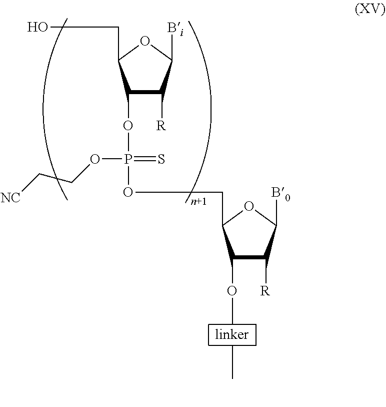
in which:
n is an integer comprised between 1 and 100, in particular 1 and 50, and in particular 5 and 40
i is an integer comprised between 1 and n+1,
B′₀ and B′ᵢ represent independently of each other a nitrogenous base, in particular:
a purine derivative, in particular adenine, guanine, 8-hydroxy guanine, inosine, or
a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,
R represents hydrogen or a hydroxyl group, 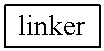
in particular chosen from:
a —CO—Z—CO— group, in which Z represents an alkyl group with 1 to 2 carbon atoms or a —CH₂—O-Ph-O—CH₂— group,
a —CO—CO— group,
a group of formula (A) or (B) below: 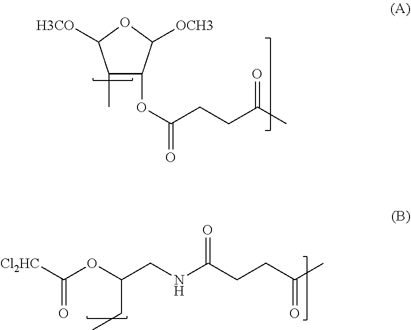 [symbol]
represents a solid support in particular chosen from CPG (Controlled Pore Glass) with long chain aminoalkyl (lcaa) of different porosities and the resins derived from polystyrene,
the reaction is followed by a reaction in basic medium, in particular with ammonium hydroxide, sodium hydroxide, methylamine, potassium carbonate, or a mixture of these bases, in order to obtain said compound of formula (V).

38. The method according to claim 32, wherein the compound prepared is further defined according to formula (VI) below: 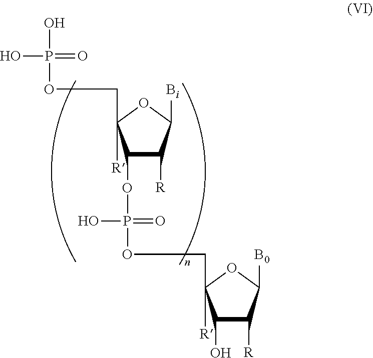
in which:
n is an integer comprised between 1 and 100, in particular 1 and 50, and in particular 5 and 40
i is an integer comprised between 1 and n,
B₀ and Bᵢ represent independently of each other a nitrogenous base, in particular:
a purine derivative, in particular adenine, guanine, 8-hydroxy guanine, inosine, or
a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
R represents:
a linear or branched —O-alkyl group comprising 1 to 4 carbon atoms, or
a —O—(CH₂)ₘ—NH₂ group, in which m represents an integer comprised between 2 and 10, or
an oxygen atom,
R′ represents:
hydrogen, or
a —(CH₂)ₚ— group in which p varies from 1 to 4,
such that when R represents an oxygen atom, R′ represents a —(CH₂)ₚ— group, and when R′ represents a —(CH₂)ₚ— group, R represents an oxygen atom, the R and R′ groups being bridged and then forming an ether ring, and
the stage of reaction involves the oxidant and a compound of formula (XVI) below: 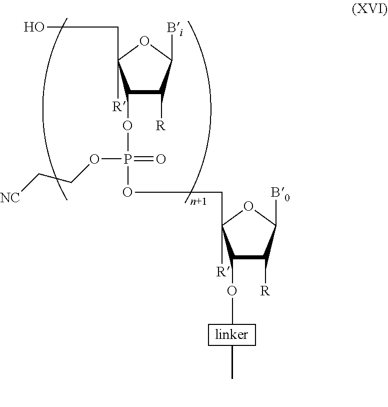
in which:
n is an integer comprised between 1 and 100, in particular 1 and 50, and in particular 5 and 40,
i is an integer comprised between 1 and n,
B′₀ and B′ᵢ represent independently of each other a nitrogenous base, in particular:
a purine derivative, in particular adenine, guanine, 8-hydroxy guanine, inosine, or
a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,
R represents:
a linear or branched —O-alkyl group comprising 1 to 4 carbon atoms, or
an —O—(CH₂)ₘ—NH₂ group, in which m represents an integer comprised between 2 and 10, or
an oxygen atom,
R′ represents:
hydrogen, or
a —(CH₂)ₚ— group in which p varies from 1 to 4,
such that when R represents an oxygen atom, R′ represents a —(CH₂)ₚ— group, and when R′ represents a group —(CH₂)ₚ—, R represents an oxygen atom, the R and R′ groups being bridged and then forming an ether ring, except in the case of the last base, where R′ represents hydrogen.
and,
Rᵢ″ represents independently a hydroxyl or thiol group, 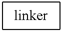
in particular chosen from:
a —CO—Z—CO— group, in which Z represents an alkyl group with 1 to 2 carbon atoms or a —CH₂—O-Ph-O—CH₂— group,
a —CO—CO— group,
a group of formula (A) or (B) below: 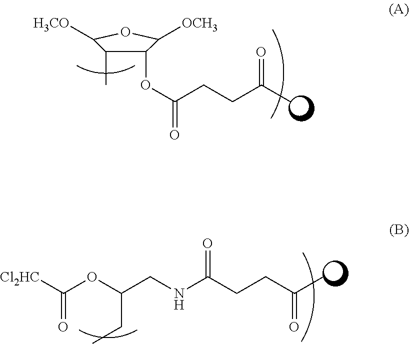 [symbol]
represents a solid support in particular chosen from CPG (Controlled Pore Glass) with long chain aminoalkyl (lcaa) of different porosities and the resins derived from polystyrene, and
the reaction is followed by a reaction in basic medium, in particular with ammonium hydroxide, sodium hydroxide, methylamine, potassium carbonate, or a mixture of these bases, in order to obtain said compound of formula (VI).

39. A method for preparing a compound of formula (XX) below: 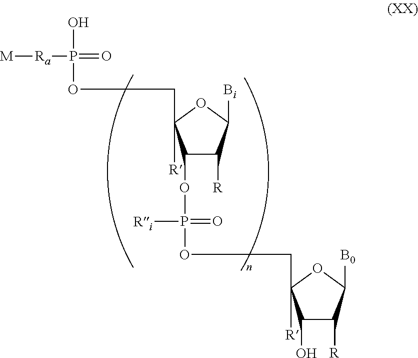
in which:
n is an integer comprised between 1 and 100, in particular 1 and 50, and in particular 5 and 40,
i is an integer comprised between 1 and n,
B₀ and Bᵢ represent independently of each other a nitrogenous base, in particular:
a purine derivative, in particular adenine, guanine, 8-hydroxy guanine, inosine, or
a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
R represents:
hydrogen, or
a hydroxyl group, or
a linear or branched —O-alkyl group comprising 1 to 4 carbon atoms, or
an oxygen atom,
R′ represents:
hydrogen, or
a —(CH₂)ₚ— group in which p varies from 1 to 4,
such that when R represents an oxygen atom, R′ represents a —(CH₂)ₚ— group, and when R′ represents a —(CH₂)ₚ— group, R represents an oxygen atom, the R and R′ groups being bridged and then forming an ether ring, and
Rᵢ″ represents independently a hydroxyl or thiol group,
Ra represents a sulphur or oxygen atom,
M represents a fluorescent probe, a peptide, an oligonucleotide or an intercalating agent,
comprising the stage of:
reaction of an oxidant chosen from the hypervalent iodine derivatives, in particular 2-iodoxybenzoic acid (IBX) and Dess-Martin periodinane (DMP), and Swern's reagent, on a compound of formula (XI) below: 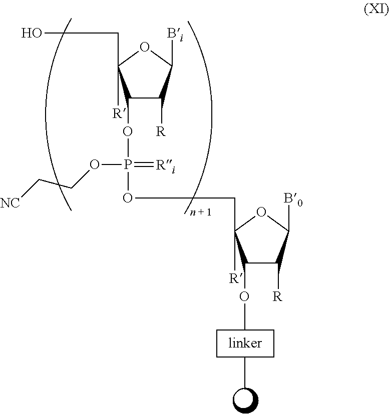
in which:
n is an integer comprised between 1 and 100, in particular 1 and 50, and in particular 5 and 40,
i is an integer comprised between 1 and n+1,
B′₀ and B′ᵢ represent independently of each other a nitrogenous base, in particular:
a purine derivative, in particular adenine, guanine, 8-hydroxy guanine, inosine, or
a pyrimidine derivative, in particular thymine, cytosine, uracil or 5-methylcytosine, 5-propynyl-uracil, pseudo-uracil, 4-thio-thymine, 2-thio-thymine, 4-thio-uracil, 5-halogeno-uracil, 5-halogeno-cytosine, 5,6-dihydro-thymine, 5-hydroxy-uracil, 5-hydroxy-cytosine, 5,6-dihydro-uracil, 5-hydroxymethyl-uracil, 5,6-dihydroxy-thymine,
said nitrogenous base being protected by at least one protective group, in particular chosen from the benzoyl (Bz), acetyl (Ac), isobutyryl (iBu), dimethylformamidine (dmf), phenoxyacetyl (Pac), 4-isopropyl-phenoxyacetyl (iPr-Pac) groups,
R represents:
hydrogen, or
a hydroxyl group, or
a linear or branched —O-alkyl group comprising 1 to 4 carbon atoms, or
an oxygen atom,
R′ represents:
hydrogen, or
a —(CH₂)ₚ— group in which p varies from 1 to 4,
such that when R represents an oxygen atom, R′ represents a —(CH₂)ₚ— group, and when R′ represents a —(CH₂)ₚ— group, R represents an oxygen atom, the R and R′ groups being bridged and then forming an ether ring, except in the case of the last base, where R′ represents hydrogen.
and,
Rᵢ″ represents independently a hydroxyl or thiol group, 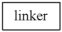
represents a linker in particular chosen from:
a —CO—Z—CO— group, in which Z represents an alkyl group with 1 to 2 carbon atoms or a —CH₂—O-Ph-O—CH₂— group,
a —CO—CO— group,
a group of formula (A) or (B) below: 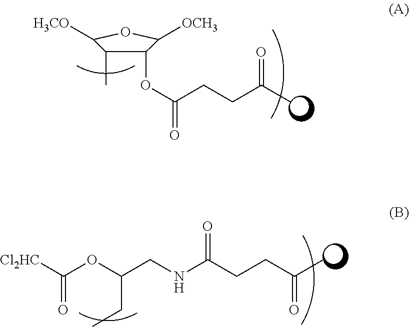 [symbol]
represents a solid support, in particular chosen from CPG (Controlled Pore Glass) with long chain aminoalkyl (lcaa) of different porosities and the resins derived from polystyrene,
followed by a reaction with a molecule M-GP, in which M represents a fluorescent probe, a peptide, an oligonucleotide or an intercalating agent, and GP represents a leaving group, in particular chosen from chlorine, bromine, iodine, the tosylate, mesylate groups, when Ra is a sulphur atom, or with a molecule M-OH or M-NH₂ after activation of the 5′-phosphate when Ra is an oxygen atom, and by a reaction in basic medium, in particular with ammonium hydroxide, sodium hydroxide, methylamine, potassium carbonate, or a mixture of these bases, said reactions with a molecule M-GP, M-OH or M-NH₂ and in basic medium being able to be carried out successively or in a single “one-pot” stage, in order to obtain said compound of formula (XX).

40. A method according to claim 32, wherein the oxidant is IBX.
